(19) United States
(12) Patent Application Publication
(10) Pub. No.: US 2010/0279418 A1
(43) Pub. Date: Nov. 4, 2010

(54) GLUCOSE METER ADAPTABLE FOR USE WITH HANDHELD DEVICES, AND ASSOCIATED COMMUNICATION NETWORK

(76) Inventors: Loren Robert Larson, Fremont, CA (US); Spencer Johnson, Palo Alto, CA (US)

Correspondence Address: GREENBERG TRAURIG, LLP (SV);IP DOCKETING, 2450 COLORADO AVENUE, SUITE 400E, SANTA MONICA, CA, 90404, US

(21) Appl. No.: 12/773,819

(22) Filed: May 4, 2010

Related U.S. Application Data
(60) Provisional application No. 61/175,397, filed on May 4, 2009.

(51) Int. Cl. G01N 33/48; G01N 31/22

(52) U.S. Cl. 436/63; 422/58

(57) ABSTRACT

Module adaptable to communicate with a suitable handheld devices or PDAs. Suitable devices include, but are not limited to, the Apple iPhone® or iPod®, Research in Motion Blackberry® smart phones, Motorola Droid smart phones, and Palm Pre smart phones. The module can be used without adding to the cost of the handheld device. This allows direct reimbursement for the replaceable meter module portion if payers choose to limit coverage for the full system, as well as the possibility of reimbursement for the entire system including the handheld device. Other solutions build the cost into the phone, which must be replaced to upgrade or replace the glucose function. Moreover, information from the glucose meter reading can be communicated from the PDA to a remote station for reporting the results. With an iPod-like approach, this could be accomplished without the need for a cellular signal or carrier, as long as a WiFi internet connection is available anywhere in the world.

### CROSS-REFERENCE

This application claims the benefit of U.S. Provisional Application No. 61/175,397, filed May 4, 2009, which application is incorporated herein by reference.

### BACKGROUND OF THE INVENTION

Diabetes mellitus is a disease characterized by elevated levels of plasma glucose. Uncontrolled hyperglycemia is associated with increased risk of vascular disease including, nephropathy, neuropathy, retinopathy, hypertension, and death. There are two major forms of diabetes. Type 1 diabetes (or insulin-dependent diabetes) and Type 2 diabetes (or noninsulin-dependent diabetes). The American Diabetes Association has estimated that approximately 6% of the world population has diabetes. The goal of diabetic therapy is to maintain a normal level of glucose in the blood. The American Diabetic Association has recommended that diabetics monitor their blood glucose level at least three times a day in order to adjust their insulin dosages and/or their eating habits and exercise regimen. While, glucose tests can only measure a point in time result and do not provide an overall assessment of glycemic control over a period of time, it is an important tool in diabetes care management.

Integrated cell phones with glucose testing capabilities have been developed. However, a significant drawback to these integrated devices is that they do not provide the opportunity to upgrade glucose hardware or meter functionality without replacing the entire phone. Moreover, if either the phone or glucose testing functionality is damaged, the entire device must be replaced to regain full functionality. In addition, for example, the iPhone® and iPod® Touch offer superior graphics and data display capability potential, along with the flexibility of unlimited applications to manage data and communications.

Other efforts to develop integrated treatment system with a glucose meter include, for example, insulin pump and wrist strap controller, as well as an effort to integrate the glucose meter and a cell phone. These integrated glucose meter/cellular phone combinations are under testing and currently cost $149.00 USD retail. Testing strips are proprietary and available only through the manufacturer without reimbursement by an insurance company. These “Glugophones” are currently offered in at least three forms: as a dongle for the iPhone, an add-on pack for LG model UX5000, VX5200, and LX350 cell phones, as well as an add-on pack for the Motorola Razr® cell phone. This further limits providers to AT&T for the iPhone and Verizon for the others. Similar systems have been tested for a longer time in Finland.

### SUMMARY OF THE INVENTION

Module adaptable to communicate with a suitable handheld devices or PDAs. Suitable devices include, but are not limited to, the Apple iPhone® or iPod®, Research in Motion Blackberry® smart phones, Motorola Droid smart phones, and Palm Pre smart phones. The module can be used without adding to the cost of the handheld device. This allows direct reimbursement for the replaceable meter module portion if payers choose to limit coverage for the full system, as well as the possibility of reimbursement for the entire system including the handheld device. Other solutions build the cost into the phone, which must be replaced to upgrade or replace the glucose function. Moreover, information from the glucose meter reading can be communicated from the PDA to a remote station for reporting the results. With an iPod-like approach, this could be accomplished without the need for a cellular signal or carrier, as long as a WiFi internet connection is available anywhere in the world.

The glucose device described here is an attachment module using the standard connector interface of the handheld device. A single module could be used on multiple handheld devices, saving cost. This flexibility also means that the module could be used with a handheld device, such as an iPod, in the gym, or with a handheld device, such as an iPhone, in the office, etc. Since it is detachable, it does not require extra space or size in the handheld devices itself—it is only attached when a reading is required. It also does not add cost to the handheld device hardware, unlike the integrated units. The functionality of the module could range from a simple electronic interface to the strip (using the handheld device to do all calculations, data processing, display, and communications with health care providers or data services) to an interface plus glucose calculation engine (where the module delivers an answer, and the handheld device provides further data processing, display, and communications with health care providers or data services) to a fully contained meter with a small display, using the handheld device for much richer data processing, display, and communications.

An aspect of the disclosure is directed to an apparatus for use to determine blood glucose levels. The apparatus comprises: an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions. Components, such as the logic apparatus and detector can be positioned within a suitable housing or can be configured to be engaged to functionally form a housing. The apparatus is typically handheld. A display screen adapted and configured to display at least one of instructions or measurement results can also be provided. A data processor can be adapted to determine a blood glucose value from a measurement.

Another aspect of the disclosure is directed to a method for detecting the blood glucose levels. The method comprises: obtaining a sample from a mammal; applying the sample to a test strip wherein the test strip is inserted into an aperture adapted and configured to receive the strip in an apparatus further comprising a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions; and determining a glucose level from the sample; communicating the glucose level to a handheld apparatus in communication with the blood glucose apparatus. Additional method steps can include, for example, one or more of, instructing a device with mobile communication functionality to contact one or more of an emergency service agency, doctor, and caregiver; displaying results of a the blood glucose measurement; and storing the measurement results on a memory device.

Still another aspect of the disclosure is directed to a networked apparatus for determining blood glucose. The networked apparatus comprises: a memory; a processor; a communicator; a display; and an apparatus for detecting a blood glucose level comprising an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions.

Still another aspect is directed to communication system. The communication system comprises: an apparatus for detecting blood glucose level comprising an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions; a server computer system; a measurement module on the server computer system for permitting the transmission of a measurement from a system for detecting blood glucose levels over a network; at least one of an API engine connected to at least one of the system for detecting blood glucose levels and the device for detecting blood glucose levels to create an message about the measurement and transmit the message over an API integrated network to a recipient having a predetermined recipient user name, an SMS engine connected to at least one of the system for detecting blood glucose levels and the device for detecting blood glucose levels to create an SMS message about the measurement and transmit the SMS message over a network to a recipient device having a predetermined measurement recipient telephone number, and an email engine connected to at least one of the system for detecting blood glucose levels and the device for detecting blood glucose levels to create an email message about the measurement and transmit the email message over the network to a recipient email having a predetermined recipient email address. Additionally, the system can further comprise a storing module on the server computer system for storing the measurement on the system for detecting blood glucose levels server database. In some configurations at least one of the system for detecting blood glucose levels and the device for detecting blood glucose levels is connectable to the server computer system over at least one of a mobile phone network and an Internet network, and a browser on the measurement recipient electronic device is used to retrieve an interface on the server computer system. Additionally, a plurality of email addresses can be held in a system for detecting blood glucose levels database and fewer than all the email addresses are individually selectable from the diagnostic host computer system, the email message being transmitted to at least one recipient email having at least one selected email address. wherein at least one of the system for detecting blood glucose levels and the device for detecting blood glucose levels is connectable to the server computer system over the Internet, and a browser on the measurement recipient electronic device is used to retrieve an interface on the server computer system. A plurality of user names can be held in the system for detecting blood glucose levels database and fewer than all the user names are individually selectable from the diagnostic host computer system, the message being transmitted to at least one measurement recipient user name via an API. Additionally, measurement recipient electronic device (e.g., smart phone, computer or glucose measurement device) is connectable directly or indirectly to the server computer system over the Internet, and a browser on the measurement recipient electronic device is used to retrieve an interface on the server computer system. Typically, the measurement recipient electronic device is connected to the server computer system over a cellular phone network. In many cases, the measurement recipient electronic device is a mobile device. An interface can also be provided on the server computer system, the interface being retrievable by an application on the mobile device. An SMS message is received by a message application on the mobile device. In some instances, a plurality of SMS messages are received for the measurement, each by a respective message application on a respective recipient mobile device. Typically, at least one SMS engine receives an SMS response over the cellular phone SMS network from the mobile device and stores an SMS response on the server computer system. Additionally, the measurement recipient phone number ID is transmitted with the SMS message to the SMS engine and is used by the server computer system to associate the SMS message with the SMS response. The server computer system can be configured to be connectable over a cellular phone network to receive a response from the measurement recipient mobile device. Additionally, the SMS message can include a URL that is selectable at the measurement recipient mobile device to respond from the measurement recipient mobile device to the server computer system, the server computer system utilizing the URL to associate the response with the SMS message. In some configurations, the system can further comprise, a downloadable application residing on the measurement recipient mobile device, the downloadable application transmitting the response and a measurement recipient phone number ID over the cellular phone network to the server computer system, the server computer system utilizing the measurement recipient phone number ID to associate the response with the SMS message; a transmissions module that transmits the measurement over a network other than the cellular phone SMS network to a measurement recipient user computer system, in parallel with the measurement that is sent over the cellular phone SMS network; and/or a downloadable application residing on the measurement recipient host computer, the downloadable application transmitting a response and a measurement recipient phone number ID over the cellular phone network to the server computer system, the server computer system utilizing the measurement recipient phone number ID to associate the response with the SMS message.

Another aspect of the disclosure is directed to a networked apparatus. The networked apparatus comprises: a memory; a processor; a communicator; a display; and an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions.

Still another aspect of the disclosure is directed to a communication system. The communication system comprises: an apparatus for detecting blood glucose level comprising an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions; a server computer system; a measurement module on the server computer system for permitting the transmission of a measurement from a system for detecting blood glucose levels over a network; at least one of an API engine connected to at least one of the system for detecting blood glucose levels and the device for detecting blood glucose levels to create an message about the measurement and transmit the message over an API integrated network to a recipient having a predetermined recipient user name, an SMS engine connected to at least one of the system for detecting blood glucose levels and the device for detecting blood glucose levels to create an SMS message about the measurement and transmit the SMS message over a network to a recipient device having a predetermined measurement recipient telephone number, and an email engine connected to at least one of the system for detecting blood glucose levels and the device for detecting blood glucose levels to create an email message about the measurement and transmit the email message over the network to a recipient email having a predetermined recipient email address. A storing module can also be provided on the server computer system for storing the measurement on the system for detecting blood glucose levels server database. In some configurations at least one of the system for detecting blood glucose levels and the device for detecting blood glucose levels is connectable to the server computer system over at least one of a mobile phone network and an Internet network, and a browser on the measurement recipient electronic device is used to retrieve an interface on the server computer system. Additionally, a plurality of email addresses are held in a system for detecting blood glucose levels database and fewer than all the email addresses are individually selectable from the diagnostic host computer system, the email message being transmitted to at least one recipient email having at least one selected email address. In some configurations, at least one of the system for detecting blood glucose levels and the device for detecting blood glucose levels is connectable to the server computer system over the Internet, and a browser on the measurement recipient electronic device is used to retrieve an interface on the server computer system. A plurality of user names can be held in the system for detecting blood glucose levels database and fewer than all the user names are individually selectable from the diagnostic host computer system, the message being transmitted to at least one measurement recipient user name via an API. Moreover, the measurement recipient electronic device is connectable to the server computer system over the Internet, and a browser on the measurement recipient electronic device is used to retrieve an interface on the server computer system. The measurement recipient electronic device can be connected to the server computer system over a cellular phone network, such as where the measurement recipient electronic device is a mobile device. Additionally, an interface on the server computer system, the interface being retrievable by an application on the mobile device. The SMS message can be received by a message application on the mobile device and, in at least some instances, a plurality of SMS messages are received for the measurement, each by a respective message application on a respective recipient mobile device. At least one SMS engine can be configured to receive an SMS response over the cellular phone SMS network from the mobile device and stores an SMS response on the server computer system. A measurement recipient phone number ID is transmitted with the SMS message to the SMS engine and is used by the server computer system to associate the SMS message with the SMS response. A server computer system is connectable over a cellular phone network to receive a response from the measurement recipient mobile device. The SMS message can includes, for example, a URL that is selectable at the measurement recipient mobile device to respond from the measurement recipient mobile device to the server computer system, the server computer system utilizing the URL to associate the response with the SMS message. The system can further include a downloadable application residing on the measurement recipient mobile device, the downloadable application transmitting the response and a measurement recipient phone number ID over the cellular phone network to the server computer system, the server computer system utilizing the measurement recipient phone number ID to associate the response with the SMS message; a transmissions module that transmits the measurement over a network other than the cellular phone SMS network to a measurement recipient user computer system, in parallel with the measurement that is sent over the cellular phone SMS network; a downloadable application residing on the measurement recipient host computer, the downloadable application transmitting a response and a measurement recipient phone number ID over the cellular phone network to the server computer system, the server computer system utilizing the measurement recipient phone number ID to associate the response with the SMS message.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a perspective view of a handheld device suitable for use with the invention;

FIG. 1B is a depiction of a testing strip suitable for glucose testing;

FIG. 2A is a depiction of a handheld device in communication with an attachable glucose monitor;

FIG. 2B is a depiction of a handheld device in communication with an attachable glucose monitor wherein the monitor has a read-out screen;

FIGS. 3A-B are schematic block diagrams of devices for diabetes monitoring;

FIG. 4 is a flow chart illustrating method steps; and

FIG. 5A is a block diagram showing a representative example of a logic device through which a dynamic modular and scalable system can be achieved; and

FIG. 5B is a block diagram showing the cooperation of exemplary components of a system suitable for use in a system where dynamic data analysis and modeling is achieved.

### DETAILED DESCRIPTION OF THE INVENTION

Currently there is no way for a handheld device to directly interface with a blood glucose meter thereby providing the potential for a rich data processing, display, and communications capabilities of the handheld device (e.g., iPhone and iPod Touch). These configurations provide the advantage of: flexibility of functionality and reimbursement coverage; ability for module provider to upgrade module over time without user having to upgrade phone; ability for any diabetes company to develop a module to work with the handheld device. These benefits also limit the cost of phone hardware and provide limited capability to develop and use future applications.

The present invention relates to glucose monitoring systems and methods, and more particularly to a system that adapts to engage a handheld device and which is configured to monitor the amount and rate of change of glucose in a patient, communicating the results to an easy-to-read display of such monitored information.

### I. DEVICE PLATFORM/INTERFACE

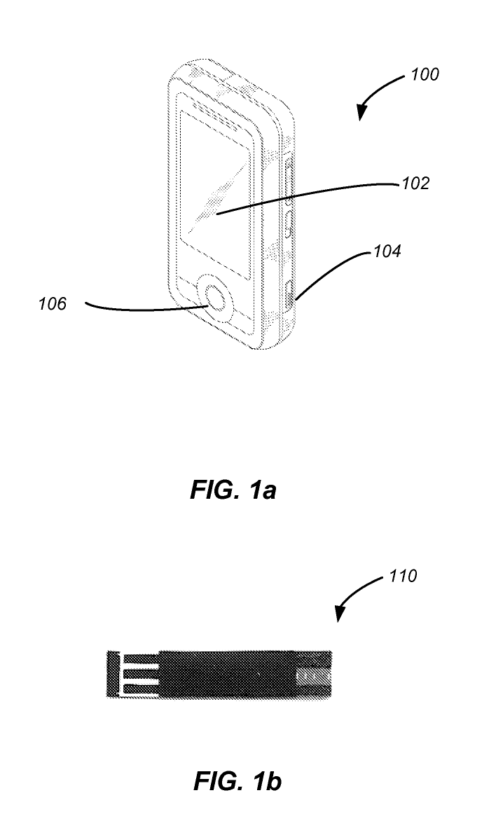

FIG. 1a illustrates a suitable smart phone device or handheld device 100 suitable for use in the system described. The handheld device 100 has a touch screen 102 and ports 104 suitable for use as, for example, data import and export, and buttons 106. However, as will be appreciated by those in skill in the art, a device which provides a keyboard could also be used without departing from the scope of the invention. FIG. 1b illustrates a standard glucose test strip 110, such as those currently available from J&J and Abbot Diabetes Care.

A standard connector interface adapted to communicate with the handheld device 100, such as a iPhone/iPod dock connector can be used to achieve communication of information between the handheld device and, for example, a measurement device or other peripheral device.

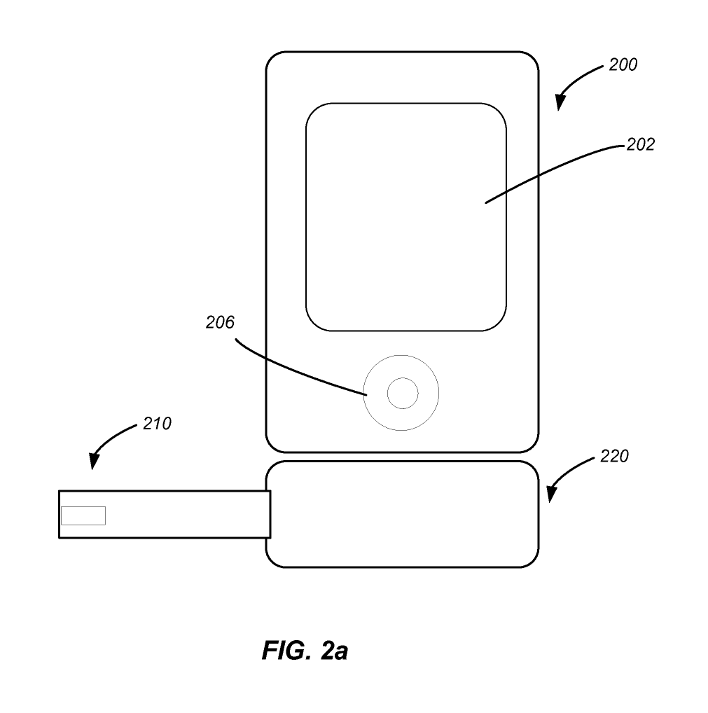

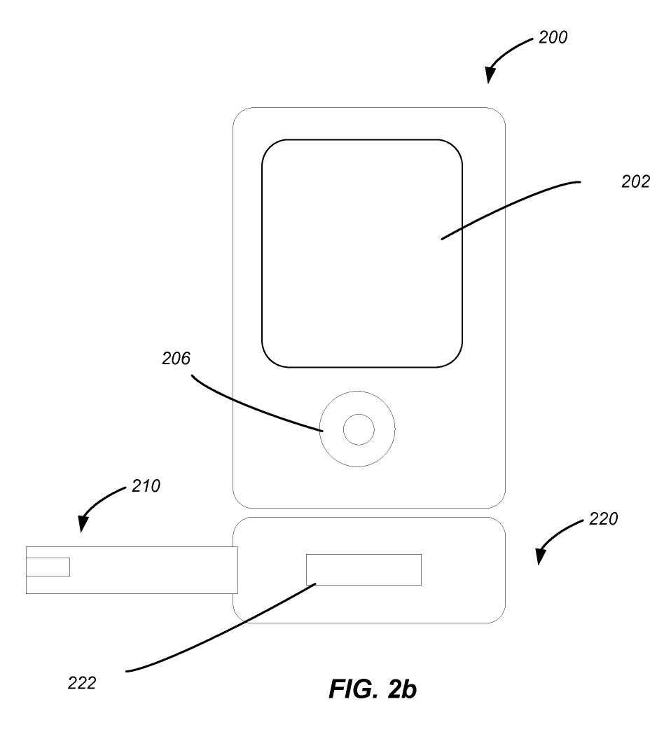

As illustrated in FIGS. 2a and 2b, the handheld device 200 is adapted and configured to engage the glucose measurement device 220. The handheld device and glucose measurement device connection can be wireless (e.g., Blue Tooth), or wired. The device 200 is illustrated to include a touch screen 202, ports, 204, and buttons 206. A test strip 210 is inserted into an aperture or channel configured to receive the strip. An aperture for receiving the test strip could, for example, be positioned on the glucose meter from a side of the measurement device that does not engage the handheld device, as illustrated in FIGS. 2. In the configuration shown in FIG. 2b, a display 222 is also provided. The test strip 210 typically contains a chemical for detecting glucose, such as glucose oxidase. This chemical reacts with the glucose in the blood sample provided by the user to create gluconic acid. Gluconic acid then reacts with, for example, ferricyanide, to create ferrocyanide. Once the ferrocyanide is created, the measurement device 220 runs an electronic current through the blood sample on the strip 210. This current is then able to detect the ferrocyanide and determine how much glucose is in the sample of blood on the test strip 210. That number is then relayed on the screen of the measurement device or on the handheld device connected to the measurement device.

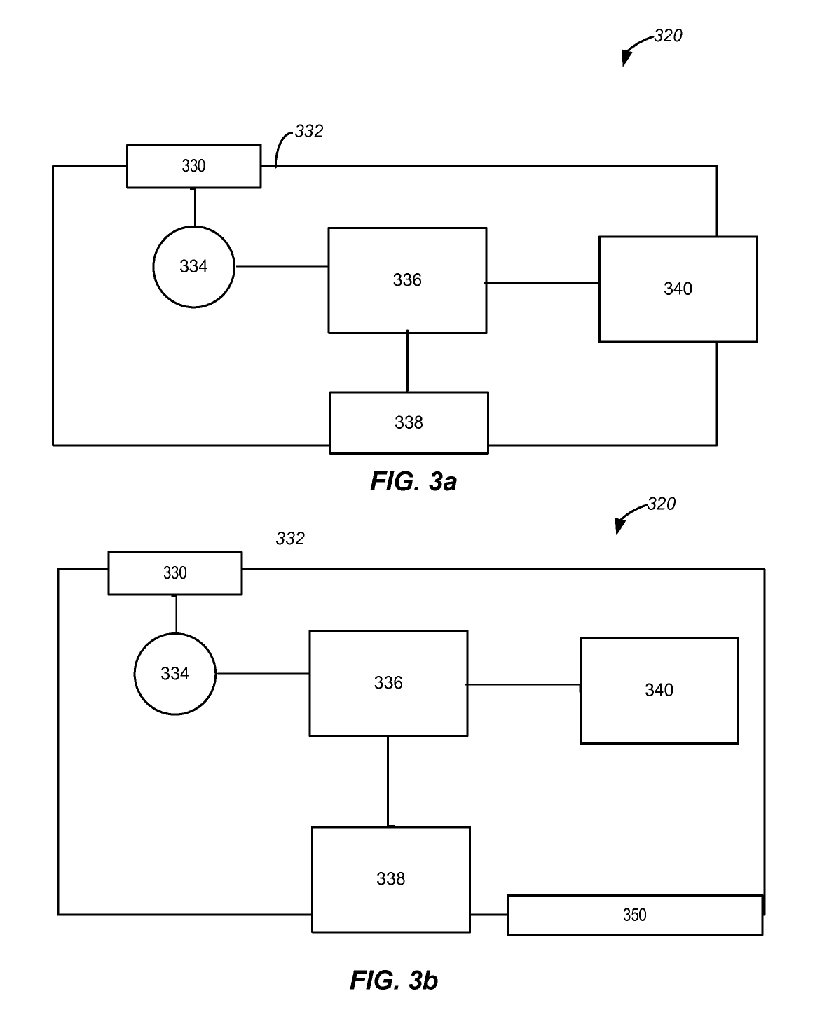

FIG. 3A-B is an illustration of a measurement device 320. A power source 330, which may be removable, adapted and configured to provide power to the system, is shown. The power source 330 can be removable, rechargeable, or fixed (as in the case of a power cord). Suitable power sources include, but are not limited to, batteries. The power source 330 may be activated by a control button 334. Moreover, power can come from an auxiliary device that the measurement device is connected to such as a computer or mobile phone. Additionally, a microcontroller can be provided on the device in order to facilitated manipulation and analysis of the information obtained from the sample. Alternatively, the information can be transmitted to a secondary device for manipulation.

An electromagnetic data storage device 336 (such as ROM, RAM, digital data storage, etc., such as in devices where data processing is provided) can be provided which stores instructions for operation of the measurement device. A memory, flash memory, and/or a full chip set or integrated circuit can be provided that interfaces (such as universal serial bus (USB) with the device. For the purposes of this disclosure a computer readable medium stores computer data, which data can include computer program code that is executable by a computer, in machine readable form. Computer readable medium may comprise computer readable storage media, for tangible or fixed storage of data, or communication media for transient interpretation of code-containing signals. Computer readable storage media includes physical or tangible storage (as opposed to signals) and includes without limitation volatile and non-volatile, removable and non-removable storage media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

A port or channel 340 is provided for interfacing with the test strip (shown above). Additionally, input buttons 338 can be provided that enable a user to input information into the device itself. In some configurations a display 350 is provided. Suitable displays include, for example, liquid crystal displays (LCD).

As will be appreciated by those skilled in the art, the system can be contained within a suitably designed housing 332 or the components can be configured such that the components are interconnected in such a way as to function as a housing. A channel or port 340 is provided which is adaptable and configurable to receive a commercially available glucose test strip (shown in FIG. 2). A blood sample placed in fluid communication with the test strip which then determines the amount of ferrocyanide by measuring an electrical current.

As will be appreciated by those skilled in the art, connectivity can also be provided which enables the system to send the information to a printer, or a network. Connectivity can be, for example, wirelessly via the internet as well as via suitable connection ports.

### II. GLUCOSE MEASUREMENTS

Electrochemical glucose monitors consist of a disposable sensor strip (110, in FIG. 1) that is made with multiple layers of conductive and reactive components, including enzymes to catalyze the reaction of the blood glucose in proximity to electrodes which capture and carry the generated current through conductors to the measurement electronics. Examples include, but are not limited to LifeScan's OneTouch Ultra® series, or Abbott's Therasense FreeStyle® series, or Roche's Accu-Chek®. FIG. 2 illustrates the OneTouch Ultra ® sensor strip, with 3 electrical contact connections on the left and blood application capillary on the right.

The module adaptor is adapted for measuring blood glucose values and for generating blood glucose data in response to measuring said blood glucose values. As discussed more fully below, the module adaptor can be configured such that it is connected to a glucose measuring apparatus. Moreover, the monitor can be adapted and configured such that it is capable of receiving, storing and evaluating data. Examples include: (a) receiving and storing blood glucose data, (b) receiving and storing physician-supplied data, (c) prompting and receiving patient input into the monitor means at periodic times of patient data relating to diet, exercise, emotional stress and symptoms of hypoglycemia and other illness experienced by the patient during a preceding time period, (d) receiving and storing the patient data supplied by the patient, and (e) generating recommendations relative to patient insulin dosage based at least in part upon the received blood glucose data, physician data and patient data.

### III. METHODS

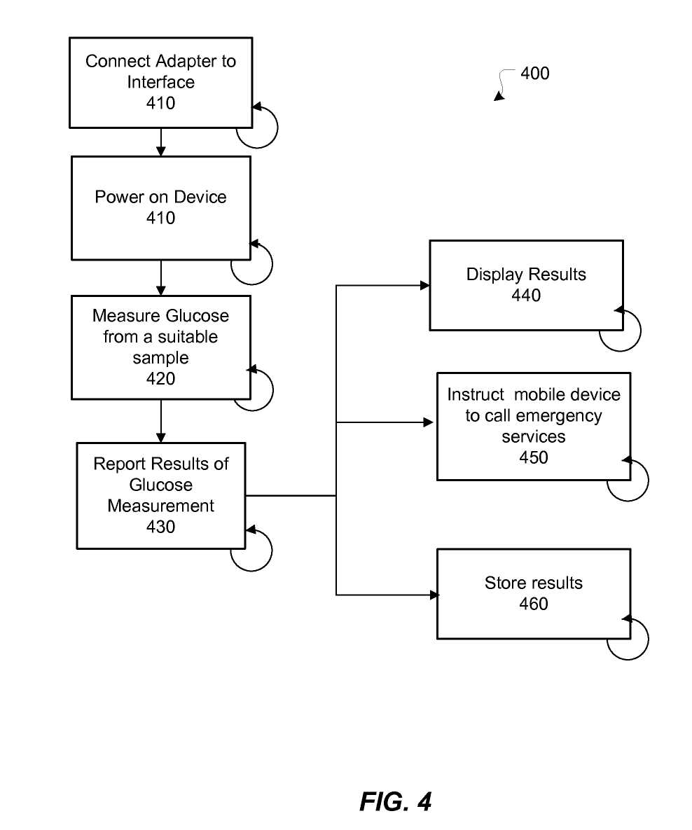

Typical Use Steps are illustrated in FIG. 4: Typical use steps include connecting a glucose measurement device to handheld device 410, e.g., through standard dock interface, a cable, or Blue Tooth connection; once the two devices are in communication, the adaptor of the invention powers-up 420, confirms ready to insert strip; instructions may then be provided to apply blood to the test strip; thereafter the device measures glucose from the sample 430 by automatically starting, and counts down to result; reports result 440, either on handheld device 450 or the measurement device, and/or instructing the device to call emergency services 460 (e.g., 911) where the sugar level is below a pre-set or patient-specific pre-determined threshold, and/or storing the data 470 on either the measurement device or the handheld device to which the measurement device is attached.

The handheld device can then be used to chart data, graph data, sort and trend data for specifics like “pre-breakfast” etc. (standard and new custom applications are possible); handheld device can transmit sets of data to diabetes professionals for further analysis or advice. Moreover, parameters can be set for the device where transmission of data occurs when, for example, a reading exceeds a certain blood glucose threshold; a series of readings exceeds an blood glucose trend, etc. Where a patient takes a blood reading and the blood glucose level is dangerously low —thus resulting in the patient being confused and unable to make a call for emergency assistance—the device could call 911 and provide a message that the patient has a dangerously low blood glucose level and may not be able to stabilize without professional intervention.

### IV. GLUCOSE MOBILE ADAPTOR DEVICES AND COMMUNICATION NETWORKS

As will be appreciated by those skilled in the art, modular and scalable system employing one or more of the glucose measurement devices discussed above can be provided which comprises a controller and more than one glucose measurement devices. Controller communicates with each glucose measurement device over a communication media. Communication media may be a wired point-to-point or multi-drop configuration. Examples of wired communication media include Ethernet, USB, and RS-232. Alternatively communication media may be wireless including radio frequency (RF) and optical. The glucose measurement device may have one or more slots for fluid processing devices such as test strips discussed above. Networked devices can be particularly useful in some situations. For example, networked devices that provide blood glucose monitoring results to a care provider (such as a doctor) can facilitate background analysis of compliance of a diabetic with diet, medication and insulin regimes which could then trigger earlier intervention by a healthcare provider when results begin trending in a clinically undesirable direction. Additionally, automatic messages in response to sample measurements can be generated to either the patient monitoring their glucose level and/or to the care provider. In some instances, automatic messages may be generated by the system to either encourage behavior (e.g., a text message or email indicating a patient is on track) or discourage behavior (e.g., a text message or email indicating that sugars are trending upward). Other automated messages could be either email or text messages providing pointers and tips for managing blood sugar. The networked communication system therefore enables background health monitoring and early intervention which can be achieved at a low cost with the least burden to health care practitioners. Additionally, live chat or texting can be facilitated via the mobile device to enable a care provider to intervene with a user real time in response to a recent communication. The user can easily review the results on the glucose measurement device while communicating with the care giver, or other person, on the wireless communication device.

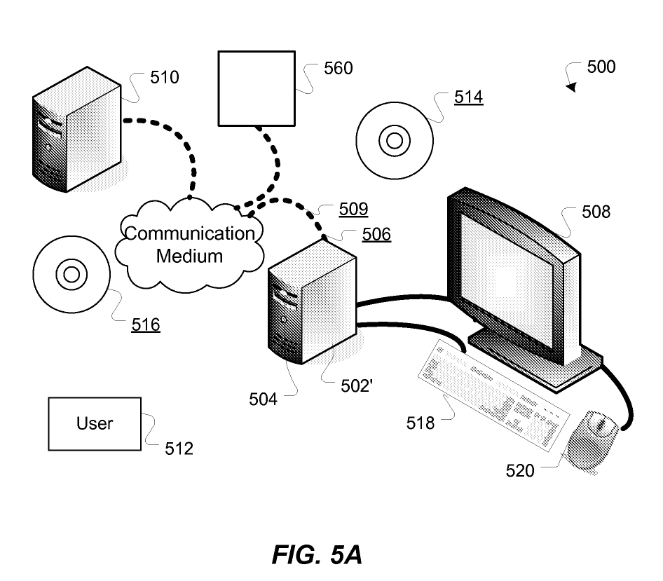

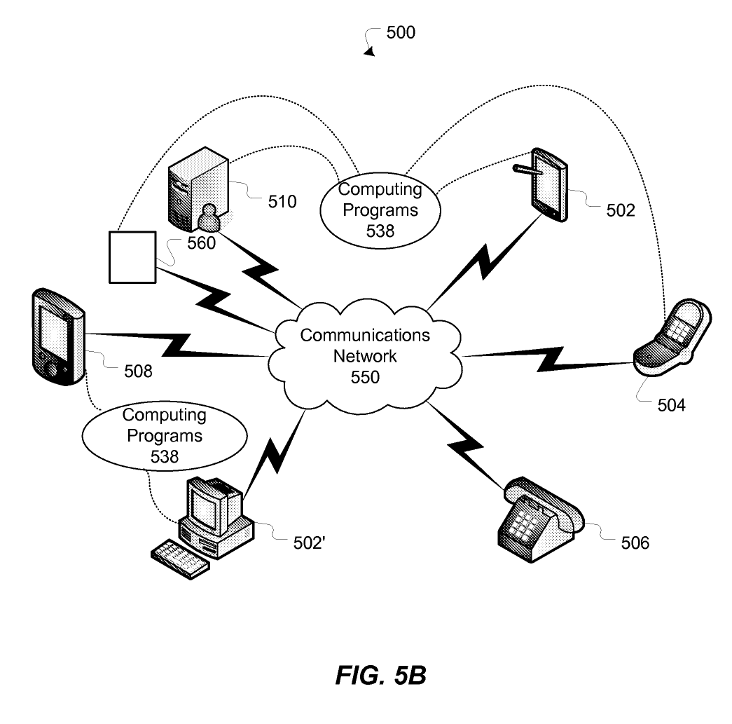

To further appreciate the networked configurations of multiple glucose measurement device in a communication network, FIG. 5A is a block diagram showing a representative example logic device through which a browser can be accessed to control and/or communication with glucose measurement device described above. A computer system (or digital device) 500, which may be understood as a logic apparatus adapted and configured to read instructions from computer readable storage media 514 which is configured to tangibly store thereon computer readable instructions and/or network port 506, is connectable to a server 510, and has a fixed media 516. The computer system 500 can also be connected to the Internet or an intranet. The system includes central processing unit (CPU) 502, disk drives 504, optional input devices, illustrated as keyboard 518 and/or mouse 520 and optional monitor 508. Data communication can be achieved through, for example, communication medium 509 to a server 510 at a local or a remote location. The communication medium 509 can include any suitable means or mechanism of transmitting and/or receiving data. For example, the communication medium can be a network connection, a wireless connection, or an internet connection. It is envisioned that data relating to the use, operation or function of the one or more glucose measurement devices (shown together for purposes of illustration here as 560) can be transmitted over such networks or connections. The computer system can be adapted to communicate with a user (users include healthcare providers, physicians, lab technicians, nurses, nurse practitioners, patients, and any other person or entity which would have access to information generated by the system) and/or a device used by a user. The computer system is adaptable to communicate with other computers over the Internet, or with computers via a server. Moreover the system is configurable to activate one or more devices associated with the network (e.g., diagnostic devices and/or glucose measurement device) and to communicate status and/or results of tests performed by the devices and/or systems.

As is well understood by those skilled in the art, the Internet is a worldwide network of computer networks. Today, the Internet is a public and self-sustaining network that is available to many millions of users. The Internet uses a set of communication protocols called TCP/IP (i.e., Transmission Control Protocol/Internet Protocol) to connect hosts. The Internet has a communications infrastructure known as the Internet backbone. Access to the Internet backbone is largely controlled by Internet Service Providers (ISPs) that resell access to corporations and individuals.

The Internet Protocol (IP) enables data to be sent from one device (e.g., a phone, a Personal Digital Assistant (PDA), a computer, etc.) to another device on a network. There are a variety of versions of IP today, including, e.g., IPv4, IPv6, etc. Other IPs are no doubt available and will continue to become available in the future, any of which can, in a communication network adapted and configured to employ or communicate with one or more glucose measurement devices, be used without departing from the scope of the disclosure. Each host device on the network has at least one IP address that is its own unique identifier and acts as a connectionless protocol. The connection between end points during a communication is not continuous. When a user sends or receives data or messages, the data or messages are divided into components known as packets. Every packet is treated as an independent unit of data and routed to its final destination—but not necessarily via the same path.

The Open System Interconnection (OSI) model was established to standardize transmission between points over the Internet or other networks. The OSI model separates the communications processes between two points in a network into seven stacked layers, with each layer adding its own set of functions. Each device handles a message so that there is a downward flow through each layer at a sending end point and an upward flow through the layers at a receiving end point. The programming and/or hardware that provides the seven layers of function is typically a combination of device operating systems, application software, TCP/IP and/or other transport and network protocols, and other software and hardware.

Typically, the top four layers are used when a message passes from or to a user and the bottom three layers are used when a message passes through a device (e.g., an IP host device). An IP host is any device on the network that is capable of transmitting and receiving IP packets, such as a server, a router or a workstation. Messages destined for some other host are not passed up to the upper layers but are forwarded to the other host. The layers of the OSI model are listed below. Layer 7 (i.e., the application layer) is a layer at which, e.g., communication partners are identified, quality of service is identified, user authentication and privacy are considered, constraints on data syntax are identified, etc. Layer 6 (i.e., the presentation layer) is a layer that, e.g., converts incoming and outgoing data from one presentation format to another, etc. Layer 5 (i.e., the session layer) is a layer that, e.g., sets up, coordinates, and terminates conversations, exchanges and dialogs between the applications, etc. Layer-4 (i.e., the transport layer) is a layer that, e.g., manages end-to-end control and error-checking, etc. Layer-3 (i.e., the network layer) is a layer that, e.g., handles routing and forwarding, etc. Layer-2 (i.e., the data-link layer) is a layer that, e.g., provides synchronization for the physical level, does bit-stuffing and furnishes transmission protocol knowledge and management, etc. The Institute of Electrical and Electronics Engineers (IEEE) sub-divides the data-link layer into two further sub-layers, the MAC (Media Access Control) layer that controls the data transfer to and from the physical layer and the LLC (Logical Link Control) layer that interfaces with the network layer and interprets commands and performs error recovery. Layer 1 (i.e., the physical layer) is a layer that, e.g., conveys the bit stream through the network at the physical level. The IEEE sub-divides the physical layer into the PLCP (Physical Layer Convergence Procedure) sub-layer and the PMD (Physical Medium Dependent) sub-layer.

Wireless networks can incorporate a variety of types of mobile devices, such as, e.g., cellular and wireless telephones, PCs (personal computers), laptop computers, tablet computers, wearable computers, cordless phones, pagers, headsets, printers, PDAs, etc. and suitable for use in a system or communication network that includes one or more glucose measurement devices. For example, mobile devices may include digital systems to secure fast wireless transmissions of voice and/or data. Typical mobile devices include some or all of the following components: a transceiver (for example a transmitter and a receiver, including a single chip transceiver with an integrated transmitter, receiver and, if desired, other functions); an antenna; a processor; display; one or more audio transducers (for example, a speaker or a microphone as in devices for audio communications); electromagnetic data storage (such as ROM, RAM, digital data storage, etc., such as in devices where data processing is provided); memory; flash memory; and/or a full chip set or integrated circuit; interfaces (such as universal serial bus (USB), coder-decoder (CODEC), universal asynchronous receiver-transmitter (UART), phase-change memory (PCM), etc.). Other components can be provided without departing from the scope of the disclosure.

Wireless LANs (WLANs) in which a mobile user can connect to a local area network (LAN) through a wireless connection may be employed for wireless communications between one or more glucose measurement devices. Wireless communications can include communications that propagate via electromagnetic waves, such as light, infrared, radio, and microwave. There are a variety of WLAN standards that currently exist, such as Bluetooth®, IEEE 802.11, and the obsolete HomeRF.

By way of example, Bluetooth products may be used to provide links between mobile computers, mobile phones, portable handheld devices, personal digital assistants (PDAs), and other mobile devices and connectivity to the Internet. Bluetooth is a computing and telecommunications industry specification that details how mobile devices can easily interconnect with each other and with non-mobile devices using a short-range wireless connection. Bluetooth creates a digital wireless protocol to address end-user problems arising from the proliferation of various mobile devices that need to keep data synchronized and consistent from one device to another, thereby allowing equipment from different vendors to work seamlessly together.

An IEEE standard, IEEE 802.11, specifies technologies for wireless LANs and devices. Using 802.11, wireless networking may be accomplished with each single base station supporting several devices. In some examples, devices may come pre-equipped with wireless hardware or a user may install a separate piece of hardware, such as a card, that may include an antenna. By way of example, devices used in 802.11 typically include three notable elements, whether or not the device is an access point (AP), a mobile station (STA), a bridge, a personal computing memory card International Association (PCMCIA) card (or PC card) or another device: a radio transceiver; an antenna; and a MAC (Media Access Control) layer that controls packet flow between points in a network.

In addition, Multiple Interface Devices (MIDs) may be utilized in some wireless networks. MIDs may contain two independent network interfaces, such as a Bluetooth interface and an 802.11 interface, thus allowing the MID to participate on two separate networks as well as to interface with Bluetooth devices. The MID may have an IP address and a common IP (network) name associated with the IP address.

Wireless network devices may include, but are not limited to Bluetooth devices, WiMAX (Worldwide Interoperability for Microwave Access), Multiple Interface Devices (MIDs), 802.11x devices (IEEE 802.11 devices including, 802.11a, 802.11b and 802.11g devices), HomeRF (Home Radio Frequency) devices, Wi-Fi (Wireless Fidelity) devices, GPRS (General Packet Radio Service) devices, 3 G cellular devices, 2.5 G cellular devices, GSM (Global System for Mobile Communications) devices, EDGE (Enhanced Data for GSM Evolution) devices, TDMA type (Time Division Multiple Access) devices, or CDMA type (Code Division Multiple Access) devices, including CDMA2000. Each network device may contain addresses of varying types including but not limited to an IP address, a Bluetooth Device Address, a Bluetooth Common Name, a Bluetooth IP address, a Bluetooth IP Common Name, an 802.11 IP Address, an 802.11 IP common Name, or an IEEE MAC address.

Wireless networks can also involve methods and protocols found in, Mobile IP (Internet Protocol) systems, in PCS systems, and in other mobile network systems. With respect to Mobile IP, this involves a standard communications protocol created by the Internet Engineering Task Force (IETF). With Mobile IP, mobile device users can move across networks while maintaining their IP Address assigned once. See Request for Comments (RFC) 3344. NB: RFCs are formal documents of the Internet Engineering Task Force (IETF). Mobile IP enhances Internet Protocol (IP) and adds a mechanism to forward Internet traffic to mobile devices when connecting outside their home network. Mobile IP assigns each mobile node a home address on its home network and a care-of-address (CoA) that identifies the current location of the device within a network and its subnets. When a device is moved to a different network, it receives a new care-of address. A mobility agent on the home network can associate each home address with its care-of address. The mobile node can send the home agent a binding update each time it changes its care-of address using Internet Control Message Protocol (ICMP).

In basic IP routing (e.g., outside mobile IP), routing mechanisms rely on the assumptions that each network node always has a constant attachment point to the Internet and that each node's IP address identifies the network link it is attached to. Nodes include a connection point, which can include a redistribution point or an end point for data transmissions, and which can recognize, process and/or forward communications to other nodes. For example, Internet routers can look at an IP address prefix or the like identifying a device's network. Then, at a network level, routers can look at a set of bits identifying a particular subnet. Then, at a subnet level, routers can look at a set of bits identifying a particular device. With typical mobile IP communications, if a user disconnects a mobile device from the Internet and tries to reconnect it at a new subnet, then the device has to be reconfigured with a new IP address, a proper netmask and a default router. Otherwise, routing protocols would not be able to deliver the packets properly.

Computing system 500, described above, can be deployed as part of a computer network that includes one or devices 560, such as glucose measurement devices disclosed herein. In general, the description for computing environments applies to both server computers and client computers deployed in a network environment. FIG. 5B illustrates an exemplary illustrative networked computing environment 500, with a server in communication with client computers via a communications network 550. As shown in FIG. 5B, server 510 may be interconnected via a communications network 550 (which may be either of, or a combination of a fixed-wire or wireless LAN, WAN, intranet, extranet, peer-to-peer network, virtual private network, the Internet, or other communications network) with a number of client computing environments such as tablet personal computer 502, mobile telephone 504, telephone 506, personal computer 502′, and personal digital assistant 508. In a network environment in which the communications network 550 is the Internet, for example, server 510 can be dedicated computing environment servers operable to process and communicate data to and from client computing environments via any of a number of known protocols, such as, hypertext transfer protocol (HTTP), file transfer protocol (FTP), simple object access protocol (SOAP), or wireless application protocol (WAP). Other wireless protocols can be used without departing from the scope of the disclosure, including, for example Wireless Markup Language (WML), DoCoMo i-mode (used, for example, in Japan) and XHTML Basic. Additionally, networked computing environment 500 can utilize various data security protocols such as secured socket layer (SSL) or pretty good privacy (PGP). Each client computing environment can be equipped with operating system 538 operable to support one or more computing applications, such as a web browser (not shown), or other graphical user interface (not shown), or a mobile desktop environment (not shown) to gain access to server computing environment 500.

In operation, a user (not shown) may interact with a computing application running on a client computing environment to obtain desired data and/or computing applications. The data and/or computing applications may be stored on server computing environment 500 and communicated to cooperating users through client computing environments over exemplary communications network 550. A participating user may request access to specific data and applications housed in whole or in part on server computing environment 500. These data may be communicated between client computing environments and server computing environments for processing and storage. Server computing environment 500 may host computing applications, processes and applets for the generation, authentication, encryption, and communication data and applications and may cooperate with other server computing environments (not shown), third party service providers (not shown), network attached storage (NAS) and storage area networks (SAN) to realize application/data transactions.

V. KITS

Bundling all devices, tools, components, materials, and accessories needed to use a measurement device to test a sample into a kit may enhance the usability and convenience of the devices. Suitable kits for glucose measurement can also include, for example, power source; test strips; wireless communication apparatus; and a glucose measurement device.

### VI. EXAMPLES

### Example 1

In some configurations, the module adaptor can be configured to connect to standard 3 conductor (or two, or more) electrochemical glucose sensor strip, and simply passes the connection through the interface with minimal circuitry or processing. The handheld device connected to the module electronically monitors the signal, and the glucose application within the handheld device contains the glucose calculation engine to convert the measured voltages/currents into a blood glucose result. The adaptor could be self powered by a rechargeable battery (e.g., from its handheld device connection) or replaceable internal battery, or could simply derive power solely off handheld device connection power.

### Example 2

In some configurations, the adapter or module itself can be configured to contain circuitry and software to power and monitor a sensor strip, and to calculate a glucose result internally before transferring this result to the handheld device. This offers some advantages in terms of completely controlling the glucose calculation, to help ensure that future changes to the handheld device or their glucose applications will not interfere with calculating a correct glucose result as detected by the adapter. Such a configuration could be configured such that it is powered by rechargeable or replaceable battery, or, as described above, off the handheld device. Moreover, permanent memory resident to the adapter could be configured to include firmware.

### Example 3

In other configurations, the adaptor or module is configured to calculate glucose values internally as described above for Example 2. Additionally, the adaptor can be configured to provide a small or simple display screen that can be used to compare/confirm the result as reported by handheld device. Also has the advantage of being a tiny stand-alone meter that can be used either attached or detached from the handheld device. For detachable use, the device would be configured to include a rechargeable or replaceable battery. However, it could also be powered off of the handheld device when it is in an attached configuration. Memory can also be provided for storing one or multiple glucose results. Stored results can then be uploaded to the handheld device in a separate step when the device is connected to a handheld device. In most configurations, the handheld device is capable of any level of complexity in glucose data reporting and storage, including simply reporting the current result in large, easy to read format, storing multiple results for later review, graphing, and otherwise trending and reporting results, and transmitting any or all of these and more to a central service or physician.

Advantages of the adapter include flexibility. As new applications come out, new data management features can be introduced. Unlike current fully integrated glucose monitoring cell phones, the adaptor module is detachable, transferable, and replaceable without purchasing a new handheld device.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A apparatus comprising:
an aperture adapted and configured to receive a glucose test strip;
a detector adapted and configured to detect at least one of a presence or amount of a substance indicative of glucose level;
a connector adapted and configured to engage the first device;
a power source; and
one or more input buttons or touch screen controls wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions.

2. The apparatus of claim 1 further comprising a housing adapted to contain the logic apparatus and detector.

3. The apparatus of claim 1 wherein the apparatus is handheld.

4. The apparatus of claim 1 further comprising a display screen adapted and configured to display at least one of instructions or measurement results.

5. The apparatus of claim 4 wherein the data processor is adapted to determine a blood glucose value from a measurement.

6. A method for detecting the blood glucose levels of a sample comprising:
obtaining a sample from a mammal;
applying the sample to a test strip wherein the test strip is inserted into an aperture adapted and configured to receive the strip in an apparatus further comprising a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions; and
determining a glucose level from the sample;
communicating the glucose level to a handheld apparatus in communication with the blood glucose apparatus.

7. The method of claim 6 further comprising the steps of instructing a device with mobile communication functionality to contact one or more of an emergency service agency, doctor, and caregiver.

8. The method of claim 6 further comprising the steps of displaying results of a the blood glucose measurement.

9. The method of claim 6 further comprising the step of storing the measurement results on a memory device.

10. A networked apparatus comprising:
a memory;
a processor;
a communicator;
a display; and
an apparatus for detecting a blood glucose level comprising an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions.

11. A communication system, comprising:
an apparatus for detecting blood glucose level comprising an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions;
a server computer system;
a measurement module on the server computer system for permitting the transmission of a measurement from a system for detecting blood glucose levels over a network;
at least one of an API engine connected to at least one of the system for detecting blood glucose levels and the device for detecting blood glucose levels to create an message about the measurement and transmit the message over an API integrated network to a recipient having a predetermined recipient user name, an SMS engine connected to at least one of the system for detecting blood glucose levels and the device for detecting blood glucose levels to create an SMS message about the measurement and transmit the SMS message over a network to a recipient device having a predetermined measurement recipient telephone number, and an email engine connected to at least one of the system for detecting blood glucose levels and the device for detecting blood glucose levels to create an email message about the measurement and transmit the email message over the network to a recipient email having a predetermined recipient email address.

12. The communication system of claim 11, further comprising a storing module on the server computer system for storing the measurement on the system for detecting blood glucose levels server database.

13. The communications system of claim 12, wherein at least one of the system for detecting blood glucose levels and the device for detecting blood glucose levels is connectable to the server computer system over at least one of a mobile phone network and an Internet network, and a browser on the measurement recipient electronic device is used to retrieve an interface on the server computer system.

14. (canceled)

15. (canceled)

16. (canceled)

17. (canceled)

18. The communications system of claim 13, wherein the measurement recipient electronic device is connected to the server computer system over a cellular phone network.

19. The communications system of claim 18, wherein the measurement recipient electronic device is a mobile device.

20. (canceled)

21. (canceled)

22. (canceled)

23. (canceled)

24. (canceled)

25. (canceled)

26. (canceled)

27. (canceled)

28. (canceled)

29. (canceled)

30. A networked apparatus comprising:
a memory;
a processor;
a communicator;
a display; and
an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions.

31. A communication system, comprising:
an apparatus for detecting blood glucose level comprising an aperture adapted and configured to receive a glucose test strip; a detector adapted and configured to detect at least one of a presence or amount of a substance indicative of glucose level; a connector adapted and configured to engage the first device; a power source; and one or more input buttons or touch screen controls, wherein the apparatus further comprises a logic apparatus adapted and configured to read instructions from a computer readable storage media associated with at least one of a first device having connectable to the Internet and the apparatus, wherein the computer readable storage media is configured to tangibly store thereon computer readable instructions;
a server computer system;
a measurement module on the server computer system for permitting the transmission of a measurement from a system for detecting blood glucose levels over a network;
at least one of an API engine connected to at least one of the system for detecting blood glucose levels and the device for detecting blood glucose levels to create an message about the measurement and transmit the message over an API integrated network to a recipient having a predetermined recipient user name, an SMS engine connected to at least one of the system for detecting blood glucose levels and the device for detecting blood glucose levels to create an SMS message about the measurement and transmit the SMS message over a network to a recipient device having a predetermined measurement recipient telephone number, and an email engine connected to at least one of the system for detecting blood glucose levels and the device for detecting blood glucose levels to create an email message about the measurement and transmit the email message over the network to a recipient email having a predetermined recipient email address.

32. The communication system of claim 31, further comprising a storing module on the server computer system for storing the measurement on the system for detecting blood glucose levels server database.

33. The communications system of claim 32, wherein at least one of the system for detecting blood glucose levels and the device for detecting blood glucose levels is connectable to the server computer system over at least one of a mobile phone network and an Internet network, and a browser on the measurement recipient electronic device is used to retrieve an interface on the server computer system.

34. The communications system of claim 32, wherein a plurality of email addresses are held in a system for detecting blood glucose levels database and fewer than all the email addresses are individually selectable from the diagnostic host computer system, the email message being transmitted to at least one recipient email having at least one selected email address.

35. The communications system of claim 34, wherein at least one of the system for detecting blood glucose levels and the device for detecting blood glucose levels is connectable to the server computer system over the Internet, and a browser on the measurement recipient electronic device is used to retrieve an interface on the server computer system.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)
